(19) United States
(12) Patent Application Publication
(10) Pub. No.: US 2021/0309961 A1
(43) Pub. Date: Oct. 7, 2021

(54) METHOD OF THREE-DIMENSIONAL MICROORGANISMS BIOFILMS FABRICATION

(71) Applicant: VIVAX BIO, LLC, New York, NY (US)

(72) Inventors: Vladislav Parfenov, Moscow region (RU); Yusef Khesuani, Moscow (RU); Frederico David Alencar de Sena Pereira, Vitória da Conquista (BR); Stanislav Petrov, Pushkino (RU); Pavel Karalkin, Moscow (RU); Elena Bulanova, Moscow (RU); Elizaveta Kudan, Moscow (RU); Alexander Ostrovskiy, Moscow (RU); Vladimir Mironov, Moscow (RU); Yakov Balakhovsky, New York, NY (US)

(21) Appl. No.: 16/837,148

(22) Filed: Apr. 1, 2020

(51) Int. Cl. C12N 1/20; C12N 13/00

(52) U.S. Cl. CPC C12N 1/20; C12N 2529/00; C12N 2525/00; C12N 13/00

(57) ABSTRACT

Current invention relates to biotechnology sphere and particularly describes the novel approach for non-attached biofilm-like aggregates fabrication in vitro under artificial microgravity environment conditions. Non-attached aggregates are grown under magnetic levitation conditions achieved by placing microorganisms suspension in paramagnetic nutrient medium in inhomogeneous magnetic field created by specifically developed permanent magnets. The invention method can produce biofilm-like aggregates from protozoa, fungi, microalgae, Gram-positive or Gram-negative bacteria and/or a consortium. The presented technology can be used for development of medications for treatment of chronic infections, antiseptics and solutions for surfaces, as well as in other applications requiring the use of non-attached biofilm-like aggregates model.

### BRANCH OF TECHNOLOGY

Present group of inventions relates to microbiology and more specifically to the method of three-dimensional microorganisms biofilms creation. The provided technology can be used for investigation of the properties of microorganisms, for developing of medications against chronic infections and for other applications requiring the model of non-attached biofilm-like aggregates.

### BACKGROUND OF THE INVENTION

It is generally admitted that bacteria exist in two states: single floating plankton cells and surface-bound cell aggregates so called biofilms [Costerton, J. W. et al., Annu. Rev. Microbiol 49(1):711 (1995)]. Biofilms are surface-bound three-dimensional multilayered structures formed by bacteria or other microorganisms (such as archaea (archaebacterias), fungi, microalgae, protozoa) or by their consortiums and self-produced matrix consisting of exopolysaccharides, proteins, cell-free DNA and lipids [Flemming, H. & Wingender, J. Nat Rev Microbiol 8, 623-633 (2010); Zhang R. et al. N. Biotechnol 51:21-30. (2019)]. Biofilms are usually described as structures formed on solid surface and more rarely on liquid surface [Gilbert, P et al. J Appl Microbiol, 92 Suppl, 98S-110S (2002)]. These are biofilms bounded to solid surface, they are studied better than others using various in vitro models. The most popular biofilm models are microtiter plates systems and constant flow fermenters [McBain, A. J. Advances in Applied Microbiology (2009)]. These models have played the leading role in understanding of biofilms growth and development stages and the mechanisms controlling these processes. Most studies that have established the exceptional role of biofilms in bacteria protection from antibiotics or other harmful effects were performed using in vitro biofilms models bounded to surface [Jass, J. et al. J. Ind. Microbiol. (1995)]. Biofilm formation enhances the bacterial resistance to antibiotics in several hundred-fold in comparison with one-celled plankton bacteria [Høiby, N et al. Int J Antimicrob Agents, 35 (4), 322-32 April (2010)]. The main protection mechanisms of biofilms are limited diffusion of certain antibiotics through the matrix, accumulation of antibiotic modifying enzymes in the matrix and differential metabolic status of microbial population. This also includes the population of so-called hungry bacteria creating stringent response which provides the survival of bacterial cells in difficult environments by saving resources, and also the presence of metabolically stable cells—persistors [Walters, M. C., et al. Antimicrob. Agents Chemother January; 47(1): 317-323 (2003)].

Biofilms formed by pathogenic organisms (especially bacteria) play critical role in clinical pathology. Besides the well known role in the development of hospital-acquired infections associated with biofilms formation on medical equipment, implants, catheters, etc., biofilms itself can cause chronic infections [Gominet, M et al. APMIS 125 (4), 365-375 (2017)]. Biofilms formed by bacterial pathogens are the proven factor of chronic bronchitis, otitis and rhinosinusitis development [Høsiby, N et al. Future Microbiology 5 (11), 1663-74 (2010)]. Bacteria in biofilms are protected from humoral and cell-mediated response because diffusion of soluble factors is limited by the matrix and immune cells can not pass through the biofilms [Alhede, M. et al., Microbiology 155(Pt 11):3500-8 (2009)].

Meantime, development of microscopic techniques allowed to perform the biofilms analysis in vivo in tissue samples. Many cases have illustrated that biofilms related to chronic diseases are not bounded to human tissues but levitate in body fluids [Alhede, M. et al. PLoS One 6(11): e27943 (2011)]. This difference points out the limitations of in vitro biofilm models [Roberts, A. E. L. J Mol Biol, 427 (23), 3646-61 (2015)]. Although the morphological features of biofilms formed on abiotic surfaces and in vivo were similar, the development mechanisms of surface-bound biofilms and non-attached biofilm-like aggregates (mostly initial stages) may if not differ but at least have different nature. The importance of researches considering non-attached biofilm-like aggregates has increased due to description of aggregates formed by bacteria grown under microgravity conditions [Zea, L. et al. Front. Microbiol. 8, 1598 (2017)].

Whereas in vitro models of surface-bound biofilms can not precisely simulate processes occurring in biofilms formed by microorganisms in real-life conditions, it is still relevant for scientists working in microbiology and related fields to create biofilm models as close to real life as possible.

### DISCLOSURE OF THE INVENTION

The object of the invention is development of the method and the creation of three-dimensional biofilm-like aggregates non-attached to substrate and which properties are close to microorganisms properties in biofilms formed under real-life conditions.

Therefore, the new approach for in vitro microorganisms aggregates fabrication (including bacteria) is suggested. The aggregates non-attached to substrate are formed by microorganisms cultivated under magnetic levitation. The levitation is achieved by placing the microorganisms on paramagnetic substrate in inhomogeneous magnetic field created with specially designed permanent magnets detailed in the following article: Parfenov V. A. et al. Scaffold-free, label-free and nozzle-free biofabrication technology using magnetic levitational assembly, 10(3):034104. Biofabrication (2018).

The fabrication method of biofilm-like microorganisms aggregates non-attached to substrate, including their culturing under magnetic levitation conditions in inhomogeneous magnetic field is provided in further details.

The method is characterized by that microorganisms are cultured in the central part of inhomogeneous magnetic field with the lowest field intensity parameters in some embodiments of the invention.

In some embodiments the inhomogeneous magnetic field is created via magnetic system consisted of at least two annular neodymium magnets facing analogous poles.

In some embodiments the inhomogeneous magnetic field is created via Bitter magnet.

In some embodiments microorganisms are represented as protozoa, fungi, microalgae, gram-positive or gram-negative bacteria, and/or their consortiums.

In some embodiments microorganisms are immersed in inhomogeneous magnetic field in cultivation vessel in suspension on paramagnetic substrate (which is the substrate with paramagnetic for cultivation of microorganisms).

In some specific embodiments paramagnetic properties of the substrate are provided by the presence of gadolinium (Gd³⁺).

In some specific embodiments gadolinium is added to the substrate in the form of gadobutrol.

Microorganisms are cultivated under magnetic levitation conditions in the inhomogeneous magnetic field during the period of time required for biofilm-like aggregates fabrication. In some embodiments microorganisms are cultivated until the first signs of aggregates fabrication, in other embodiments microorganisms are cultivated until fabrication of stable biofilm-like aggregates depending on purposes. The cultivation duration also depends on the microbial species and strain or its consortium. The duration may vary from minutes and hours to days, weeks or even more.

In some embodiments the cultivating environment is selected according to the microbial species and strain or its consortium that can provide optimal parameters for cultivation process itself (growth and distribution). In other embodiments the suboptimal cultivating environment can be chosen to study and analyse the effect of the environment on biofilm-like microorganisms aggregates fabrication.

In some embodiments test compounds, biologically active substances, medications and/or mixtures (e.g., test medications, antiseptics, etc.) can be added to the paramagnetic substrate to study its influence on biofilm-like microorganisms aggregates fabrication.

The selection of the cultivation parameters and the medications (as it is mentioned above) can be carried out independently. It can also vary during the cultivation process if it is necessary.

In some specific embodiments microorganisms can be cultivated in closed tube or closed syringe.

In some embodiments surplus of paramagnetic substrate can be added to the vessel with cultivated microorganisms during their growth process. In some specific embodiments there can be continuous income of paramagnetic substrate to the vessel with cultivated microorganisms during their growth process.

In some embodiments microorganisms are preliminary cultivated (before their placement in magnetic levitation conditions in the inhomogeneous magnetic field) in the paramagnetic substrate during the time required for their adaptation to the culture medium. In some specific embodiments the preliminary cultivation time can vary from 0 hours to several days, the most optimal period is 1 to 24 hours and even more preferable period is 5 to 12 hours.

The goal is also achieved by fabrication of three-dimensional biofilm-like microorganisms aggregates obtained by the method described above.

In some specific embodiments biofilm-like microorganisms aggregates of the present invention are formed by such microorganisms as protozoa, fungi, microalgae, Gram-positive or Gram-negative bacteria and/or its consortiums.

The following technical results can be achieved due to of invention embodiment:
the novel method of non-attached microorganisms biofilms (biofilm-like aggregates) in vitro fabrication is developed;
the created biofilm-like aggregates are not attached to any surfaces of other substrates and have three-dimensional structure formed by microorganisms (including bacteria) and extracellular matrix;
the created biofilm-like aggregates are similar by its properties to biofilms. Its properties are:

(i) biofilm-like aggregates fabrication results from reproduction and growing of microorganisms, this aspect is similar to microcolonies formation as the key stage of biofilm fabrication;

(ii) created aggregates are three-dimensional structures formed by microorganisms and extracellular matrix, whereas, extracellular matrix is the microorganisms product and it is formed not by substrate proteins but due to their growth and development
fabricated biofilm-like aggregates has high stability and survivability and they are suitable for use in testing and development of medications against infections caused by microorganisms (when pathogenesis involves biofilm formation; chronic and non-curable infections), development of antiseptics and/or solutions for surfaces, and any other applications that require a biofilm models or non-attached biofilm-like aggregates. The testing results for antibiotics, other antimicrobial agents and medications from such substrates are much closer to the in vivo models in comparison to two-dimensional biofilm-like aggregates;
suggested method can be used in real-life conditions for modeling of microorganisms growth in micro-gravity environment;
biofilm-like aggregates of almost any required size can be created via the suggested method; moreover, macroscopic size of bacterial aggregates allows to monitor the aggregates growth and development processes in real time, and the influence of various conditions and tested medications on their development.
suggested method is suitable for scaling via continuing
proposed method is suitable for its scaling by providing constant flow of paramagnetic culture medium.

Detailed Specification

Wide range of in vitro models for bacteria levitation has been described previously. Thus, the superconducting magnet was used for creating conditions of diamagnetic levitation. This magnet produced high-gradient high-intensity magnetic field (18 T) [Dijkstra, C. E. et al. Journal of the Royal Society Interface (2011)]. The increased growth of bacteria was observed in such conditions but there was no aggregates fabrication.

Sriramulu et al. describes in the article (J. Med. Microbiol. 54 (Pt 7), 667-76 (2005)) the modelling of P. aeruginosa behavior in the alveolus of patients with cystic fibrosis (CF) that is the hereditary disorder caused by mutations leading to chloride secretion deficiency and further thick congestive mucus accumulation in pulmonary alveolus. In this research the pathogens were cultivated in highly viscous synthetic mucous medium ASM including mucin and cell-free DNA. P. aeruginosa has grown in these conditions, formed stable biofilm-like structures non-attached to the polystyrene or glass surface. They imitated structures observed in vivo, however, microorganisms have grown in solid microcolonies attached to the sputum components. The obtained biofilm-like structures are the example of two-dimensional biofilms. The performed antibiotics analysis has shown that their overestimated efficacy was not confirmed later in clinical trials.

Rotating Wall Vessel developed in Johnson Space Center (Houston, Tex.) is one of the most popular models of microgravity models in the world. It provides non-attached bacterial aggregates fabrication under conditions of constant rotation[Nickerson, C. A., et al. Mol. Biol. Rev. (2004)]. However, Rotating Wall Vessel can not provide stable three-dimensional biofilm-like microorganisms aggregates due to turbulence and hemodynamic stress resulting from constant fluid movement in the vessel.

Magnetic levitation is widely used in industry and researches to create specific conditions when the object is suspended without any support except magnetic fields which are opposite to gravity [Sadiku, M. & Akunjuobi, C. IEEE Potentials 25, 41-42 (2006)]. The combination of permanent magnets and diamagnetics or superconductors is commonly used to achieve such effect. The magnetic levitation is used in biology in microfluid researches for developing of cell collection and analysis systems [Wang, Z. M. et al. Sci. Rep. (2016)].

The method suggested by the invention is based on bacteria's growth in the magnetic levitation conditions when magnetic force compensates gravity force. Magnetic levitation is achieved by placing growing bacterial culture in magnetic trap. The capture effect is based on the differential magnetic properties of diamagnetic cells and paramagnetic gadolinium-containing medium in inhomogeneous magnetic field. Bacteria reunites in the area with the magnetic field of highest intensity, while the medium is situated in the magnetic field of lowest intensity. Hereinafter, the detailed description of the method will be presented. The method itself enables to obtain in vitro non-attached biofilm-like microorganisms aggregates and it has shown that such aggregates have all the main characteristics of biofilms.

### BRIEF DESCRIPTION OF FIGURES

FIG. 1.

A—The scheme of magnetic bioprinter: 1—body, 2—magnetic block, 3—peephole (observation window), 4—place for cuvette insertion;

B—Direction of cuvette installing in bioprinter;

C—Combination of permanent magnets considering magnetic field gradient: 1—two magnetic rings with connected poles of same polarity, 2—metal box, 3—fixation of the box and the lid;

D—Inner part of bioprinter where the cuvette is located;

E—Magnetic field created in the bioprinter. The area where the aggregates fabrication proceeds in magnetic trap is marked with “1”.

FIG. 2. Bacteria behavior in magnetic levitation:

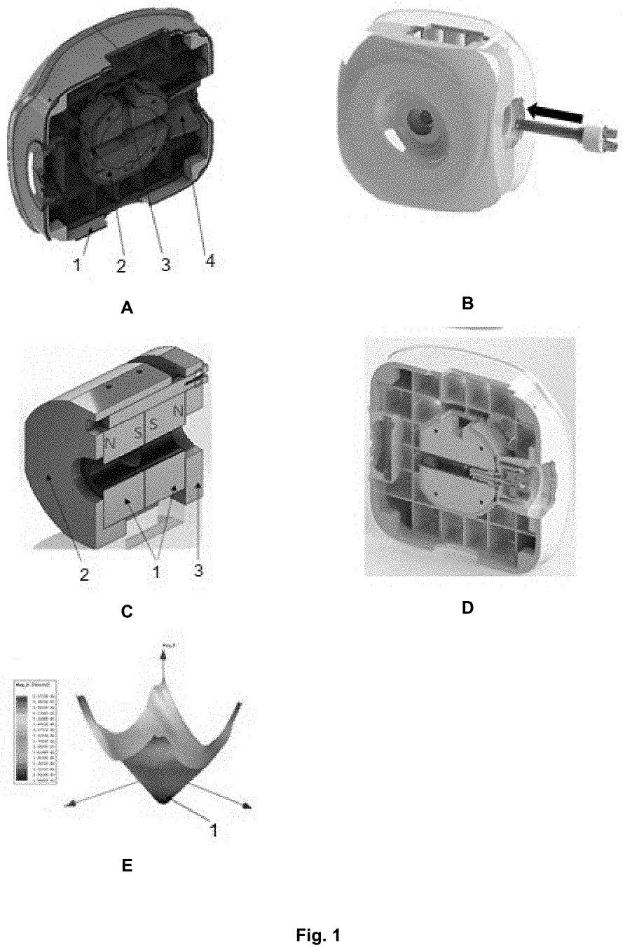

A—The day before we add 0,2 M gadobutrol in E. coli culture ATCC 43890 and next day put it inside the bioprinter. The images were made through the special peephole (FIG. 1) at start (0 hours), in 9 hours and in 24 hours. Bacteria populations sizes were measured at the same time periods;

B—The day before we add 0,1 M gadobutrol in E. coli culture ATCC 43890 and next day put it inside the bioprinter. The images were made in 24 hours;

C—The day before we diluted E. coli culture ATCC 43890 at the ratio of 1:100 with 0,1M paramagnetic substrate (LB with addition of 0,1 M gadobutrol), next day the culture was placed in bioprinter and it was incubated at 37° C. for 24 hours. The images were made after cuvette extraction;

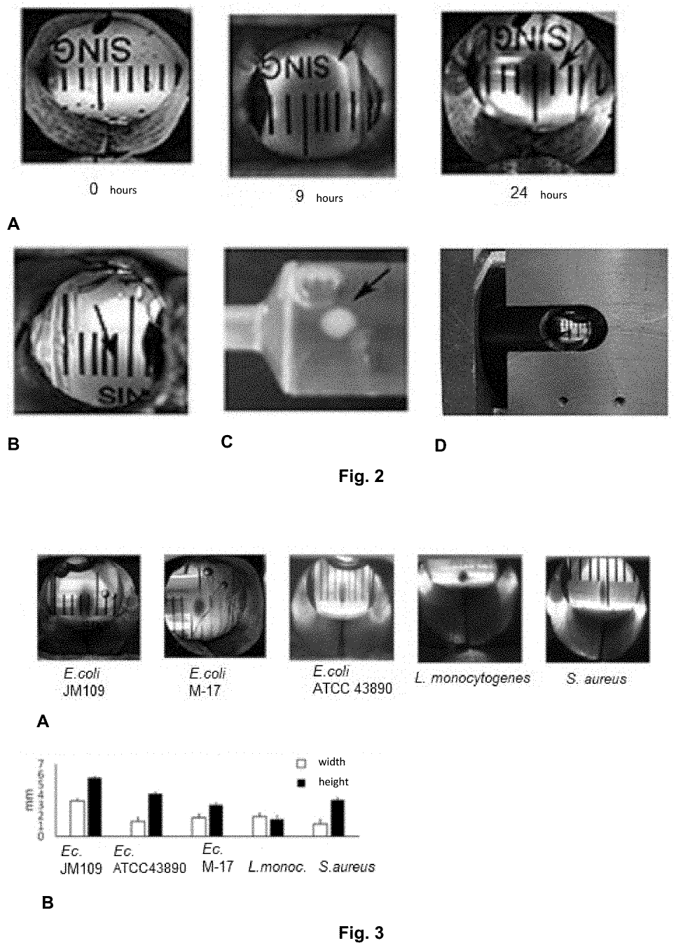

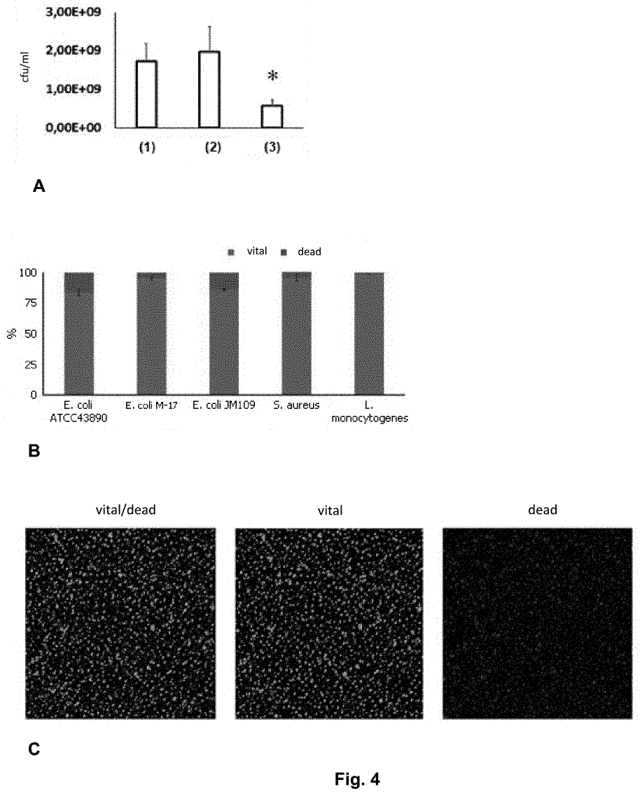

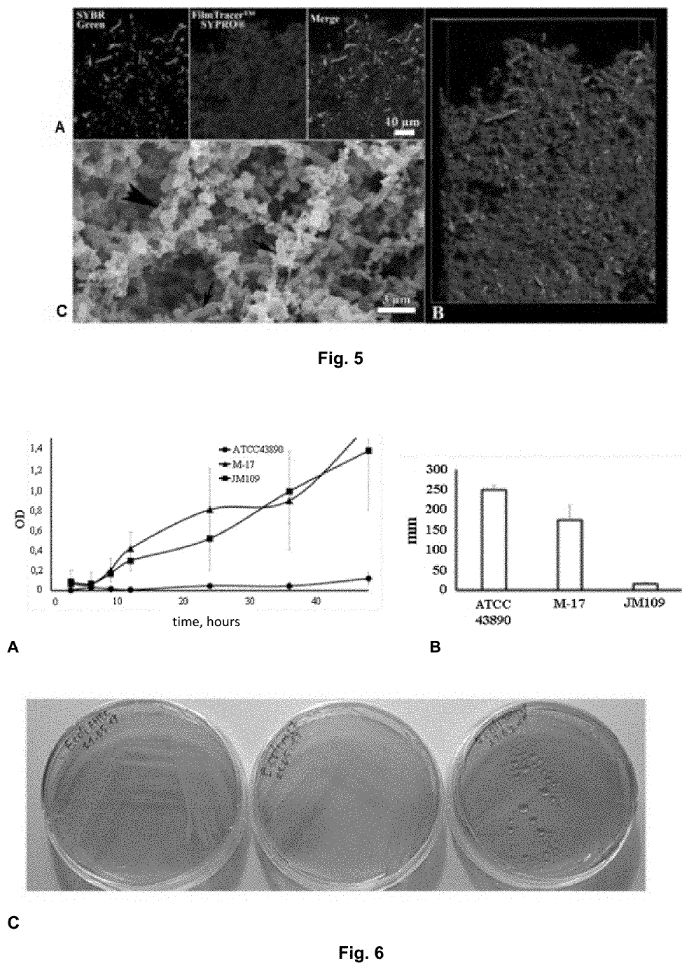

D—The paramagnetic (up to 0,2 M of gadobutrol) was added to the culture presented on FIG. 2C, it was incubated at 37° C. for 24 hours.

FIG. 3. Bacteria cultures were cultivated in magnetic levitation conditions. The day before bacteria cultures were diluted at the ratio of 1:100 with 0,2 M paramagnetic substrate and next day put it inside the bioprinter and incubate it at 37° C. for 24 hours.

A. Images of small spheres formed by various bacteria strains in bioprinter were made through the special peephole.

B. Spheres' height and width. The data is mean value±standard dispersion (SD) in three experiments.

FIG. 4. Survivability of bacteria cultivated in magnetic trap:

A. (1) E. coli culture ATCC 43890 grown on LB broth during the night, (2) bacteria culture grown on LB broth and incubated with 0,2 M of gadobutrol in magnetic bioprinter for 24 hours, and (3) bacteria culture cultivated in paramagnetic substrate containing 0,2 M of gadobutrol in magnetic bioprinter for 24 hours; bacteria were sown in decimal dilution; diagram is showing concentrations calculated in three independent experiments.

B. Relative value of vital (green) and dead (red) bacteria in cultures cultivated in magnetic trap for 24 hours.

C. The example of E. coli ATCC 43890 micrograph used for automated cell counting. The data is mean value (±SD) calculated according to 10 photos prepared in three independent experiments.

FIG. 5. Microimage of aggregates formed by E. coli ATCC 43890. Bacteria were cultivated in magnetic trap for 7 days and fixed by glutaraldehyde 2,5%. After that part of the sample was stained with SYBR Green and Film Tracer™ SYPRO® Ruby Biofilm Matrix Stain (A and B), another part was prepared for performing of SEM analysis.

A—Results of CLSM analysis has shown that bacterial aggregates are formed by bacteria and extracellular matrix; elongated and normal bacterial cells are detected;

B—3D reconstruction of the sample;

C—SEM analysis confirms the view that aggregates are formed by bacteria (small arrows) and matrix with vesicular structure (tip of the large arrow).

FIG. 6. Biofilms attached to the substrate and formed by three strains of E. coli (ATCC 43890, M-17 [symbol] JM109). They were tested in the research on microtiter plates.

A. Biofilms biomass was measured within 48 hours. ATCC 43890 strain did not form representative biofilms which could be identified by used methods.

B. Bacteria mobility was measured 16 hours after inoculation in semisolid agar. The data is mean value (±SD) from three independent experiments.

C. E. coli was grown on agar with Congo Red stain. M-17 strain (in the middle) and JM109 strain (right), but not ATCC 43890 strain (left) were coloured in red what is typical for bacteria producing curli protein.

### TERMS AND DEFINITIONS

Various terms applying to the objects of the current invention are used above and also in the description and summary of the invention. All technical and scientific terms used within this application have the same meaning for those skilled in the art, unless otherwise stated. References to the methods used in the specification refer to well known methods including any modifications to those methods and their replacement with equivalent methods known for those skilled in the art.

The terms “includes” and “including” are interpreted as “includes among other things”. These mentioned terms are not intended to be interpreted as “consists only of”.

The term “medium” (“culture medium”, “broth”) refers to any medium intended for microorganisms culturing. The medium can be solid (agar), semisolid ((gel-like) semisolid agar) and liquid. The key requirements for medium are nutritional value (thus, it has to contain all necessary substances for microorganisms), pH value optimal for specific microorganisms, buffer capacity (concentration of substance neutralising byproducts for maintenance of pH value during cultivation), isotonicity (osmotic pressure in the medium has to be the same as inside the cell), sterility (to get pure growth), sufficient amount of water (to provide osmosis and diffusion of nutrients and metabolic products), transparency, etc. For example, LB medium can be used to cultivate Escherichia coli strains.

“Paramagnetic medium” (“paramagnetic culture medium”) is the medium containing paramagnetic for microorganisms cultivation. Any compounds having paramagnetic properties (so they get magnetization in the direction of magnetic field vector when placed in external magnetic field) can be used as paramagnets. The first choice paramagnets are those which have no toxic effect on cultured microorganisms such as gadolinium salts and chelates, copper sulphate, manganese chloride, etc. according to the invention. The minimum concentration of paramagnetic is selected to ensure microorganisms levitation in inhomogeneous magnetic field. This concentration depends on the type of microorganisms, magnetic field parameters, medium composition, culture conditions, etc.

“Cultivation vessel” can be represented as cuvette, tube, vessel, bottle, syringe. In some preferred embodiments of the invention it is prescribed that the cultivation vessel can be tightly closed and sealed. In some embodiments the cultivation vessel includes the ability to add the paramagnetic medium during the cultivation process (bioreactors included). Cultivating environment for microorganisms growth (cultivation and reproduction) should be also maintained: atmosphere (gas composition, oxygen or anoxic environment), availability of nutrition in the medium, temperature, pressure, etc. Cultivating environment is chosen to be appropriate for specific microorganism species or strain (or consortium) used for biofilm-like aggregate fabrication (or inappropriate depending on the objectives). In some embodiments the cultivation vessel can have gas-permeable membrane on one side which can ensure continuous air exchanges as microorganisms are cultivated.

“Biofilm” is microorganisms biocenosis (colony) with space and metabolic structure. It is located on the interfacial surface and dipped in polymeric extracellular self-produced matrix which contains polysaccharides, proteins, nucleic acids, glycoproteins, etc. The biofilm structure is heterogeneous and dynamic. Biofilms can be formed by either a single microorganism strain or by a consortium of microorganisms like various bacteria or even various microorganisms (polymicrobic biofilms).

“Biofilm-like aggregates (structures)” are microorganisms biocenosis which are dipped in polymeric extracellular matrix and which have characteristics and properties similar to biofilms. In terms of this invention all biofilm-like aggregates are fabricated via magnetic levitation.

“Curli” is the main protein of complex extracellular matrix formed by numerous enterobacteria. This protein has amyloid origin. Curli filaments are responsible for adhesion, cell clustering and biofilms fabrication (Barnhart M. M., Chapman M. R. Annu Rev Microbiol. 60:131-47(2006)).

“Substrate” (“surface”) is the inhabitation and place for microorganisms development (bacteria, fungi, protozoa, etc.). Substrates serve as a site for microorganisms attachment and can fulfil the role of culture medium. The substrate may include both live and non-living materials and may also be solid, gel-like, liquid.

“Magnetic trap” is the geometrical arrangement of magnetic field created for limitation of movements of any object. According to the invention “magnetic trap” is formed in the central part of inhomogeneous magnetic field and it is characterised by escalation of field intensity when the object is moving from the magnetic trap in any direction. According to the invention microorganisms can not leave the magnetic trap during the process of biofilm-like aggregates fabrication. The “magnetic trap” is characterized by the minimum parameters of magnetic field intensity that ensures movement and further fabrication of levitated vital aggregates (biofilms) inside of the magnetic trap. These aggregates consist of diamagnetic microorganisms such as protozoa, fungi, microalgae, Gram-positive or Gram-negative bacteria, and/or its consortiums.

Magnetic levitation is widely used in industry and scientific researches to create conditions when the object is suspended without any support except magnetic fields which are opposite to gravity. The combination of permanent magnets and diamagnetics or superconductors is commonly used to achieve such effect. The magnetic levitation is used in biology in microfluid researches for developing of cell collection and analysis systems. This invention uses novel magnetic levitation system designed to provide scaffold-free tissue spheroids fabrication. The system was described earlier in the article Parfenov V. A. et al. Scaffold-free, label-free and nozzle-free biofabrication technology using magnetic levitational assembly, 10(3):034104. Biofabrication (2018). This system allows to fabricate levitated microorganisms aggregates. These aggregates have characteristic typical for bacterial biofilms. Though, the important difference between mechanisms of biofilm fabrication by E. coli and biofilm-like aggregates has been illustrated.

The fabrication of biofilm-like aggregates triggers extracellular matrix synthesis and accumulation processes. Consequently, it stabilizes the three-dimensional structure and gradually separates it from external magnetic field, and finally it provides high stability and survivability of the biofilm-like aggregates.

The described methods are applicable for fabrication of non-attached microorganisms aggregates such as Gram-negative or Gram-positive bacteria, protozoa, fungi, microalgae and its consortiums. They can be used in studies on biofilms non-attached to substrate, for testing and development of medications against chronic and resistant to treatment infections, at development of antiseptics and/or solutions for surfaces, and any other applications that require a biofilm models or non-attached biofilm-like aggregates. The testing results for antibiotics, other antimicrobial agents and medications from such substrates are much closer to the in vivo models in comparison to two-dimensional biofilm-like aggregates. Though, they have shown very high efficacy in many cases, it has not been confirmed later in clinical trials.

Materials and Methods

Magnetic Set

Magnetic set consists of so-called magnetic bioprinter and a cuvette filled with paramagnetic medium. The magnetic bioprinter is presented in FIG. 1A. The main elements of the bioprinter are magnetic block (2 in FIG. 1A), place for cuvette insertion (4), body (1). Magnetic bioprinter creates inhomogeneous magnetic field in the working area where the cuvette is located (FIG. 1E). Such magnetic field structure is formed via special design consisting of two magnetic rings NdFeB (N52) connected by poles of the same polarity (1 in FIG. 1C). In the illustrated (but non-limiting) embodiment the outer diameter of the magnets is 85 mm; internal diameter is 18 mm; thickness (height) is 24 mm. The magnets are assembled in such way that they are oriented towards each other by the same poles. The inhomogeneous magnetic field is created in axial bore of the magnetic set (working area). The distribution of magnetic induction values in vertical and horizontal sections is Illustrated in 3D model diagram (FIG. 1E). The peephole (observation window) (3 in FIG. 1A) allows to control the process during the experiments. The magnetic set also includes ferromagnetic shield screening magnetic field.

Operating principle of the magnetic set supposes creation of local microgravity zone which can neutralize all the forces acting on the objects. Magneto-phoretic force can appear only if the magnetic field is inhomogeneous. This causes particle movement away from the areas of intense magnetic field. Magneto-phoretic force is applicable for particles with neutral charge which have relative permeability different from underlying liquid. Thus, the effective m magneto-phoretic force F acting on the object in inhomogeneous magnetic field can be described as in the following formula:

F=2πr³μ₀μ_{f}K∇(H²),

where H is magnetic field, μ_{f}—liquid relative permeability, μₚ—particle (particles) relative permeability, μ₀—magnetic constant, and K is:

$$K = \frac{{μ}_{p} - {μ}_{f}}{{μ}_{p} + 2 {μ}_{f}} .$$

If liquid and particles relative permeability is close to 1 than magneto-phoretic force acting on the particles is approximately linear with the difference between them. Because μ>1 for paramagnetics and μ< [symbol] diamagnetics the differ μₚ−μ_{f} determines the direction of magnetic force action. As a result, objects will be pushed into the region with lowest field intensity (“magnetic trap”) due to magneto-phoretic force. Under Earthgravity conditions conditions, objects are balancing at the certain distance from the local minimum of the magnetic field.

Sterile 5 ml syringes were used as cuvettes in the experiments. The syringe was filled with the suspension of microorganisms (e.g. bacteria) in the paramagnetic culture medium. The filled syringe was placed in the magnetic bioprinter working zone.

10% or 20% (by volume) Gadovist® (Bayer) was added to the nutrient broth to create the paramagnetic culture medium. Gadovist® is a paramagnetic fluid used for clinical purposes in MRI studies. The active ingredient of Gadovist® is 1M gadobutrol([10-[2,3-dihydroxy-1-(hydroxymethyl)propyl]-1,4,7,10-tetraazacyclododecane-1,4,7-triaceto(3-)-N¹,N⁴,N⁷,N¹⁰,O¹,O⁴,O⁷]gadolinium). 10% or 20% Gadovist® is compatible with 0.1M or 0.2M gadobutrol solution respectively.

Magnetic force allows bacterial cells or any other biological particles to assemble in the center of magnetic trap (see 1 in FIG. 1E and FIG. 2). Magnetic force results from the difference in magnetic permeability of microorganisms cells that are diamagnetic and paramagnetic nutrient medium. Magnetic field goes through cells freely but the medium deforms the field when the system is placed in inhomogeneous magnetic field.

Bacterial Strains and Growth Conditions

Some bacterial strains included in experiments are presented in Table 1. The bacteria were usually cultured at 37° C. in LB (Lysogeny Broth) or BHI (Brain Heart Infection Agar) medium for Gram-negative and Gram-positive bacteria respectively.

TABLE 1
Bacterial strains

| | | | |
|---|---|---|---|
| Species/ | | General | Serotype and other |
| strain | Gram's stain | characteristics | characteristics |

Escherichia coli

| | | | |
|---|---|---|---|
| JM109 | Gram- | Laboratory | O-rough: H48; recA1 supE44 |
| | negative | strain | endA1 hsdR17 gyrA96 relA1 |
| | | | thi Δ(lac-proAB) F′ [traD36 |
| | | | proAB⁺lacl^{q}lacZΔM15] |
| M-17 | Gram- | Probiotic | O2:H6 |
| | negative | | |
| ATCC | Gram- | Virulent strain | O157:H7, Shiga-like toxin type |
| 43890 | negative | | 1 producer |

Listeria monocytogenes

| | | | |
|---|---|---|---|
| EGDe | Gram-positive | [symbol] | 1/2a |
| | | [symbol] | |
| | | [symbol] | |

Staphylococcus aureus

| | | |
|---|---|---|
| Gram-positive | Virulent strain | NA |

According to the invention this method of producing biofilm-like microorganisms aggregates non-attached to the substrate have following main steps:
microorganism culture should be placed in paramagnetic substrate which is paramagnetic nutrient medium;
microorganisms are cultivated for a time required for their adaptation to the medium; the cultivation time may vary from 0 hours to several days, particularly 1-24 hours, for example 5-12 hours depending on the microorganism species, strain, its sensitivity to paramagnetic; this step can be skipped if adaptation is not required;
then the culture is placed in central part of inhomogeneous magnetic field with the lowest field intensity, this provides magnetic levitation conditions; cultivating environment (composition of the medium, temperature, pressure, atmosphere, etc.) is selected depending on the microorganisms species and strains or its consortiums so that to provide optimal culture parameters for growth and development (depending on the objectives); the cultivation duration depends on strain, final objective of experiment and may vary from several hours to several day, weeks or even more.

The suggested method is suitable for scaling of the biofilms fabrication process by providing continuous supplement/inflow of paramagnetic medium and maintaining of optimal cultivating environment.

### EXAMPLES

### Example 1. Characteristics of Bacteria Behavior Under Magnetic Levitation Depending on Initial Parameters of Culture and Gadobutrol Concentration

To analyse the bacteria behavior in magnetic trap we have added 20% (by volume) Gadowist® in E. coli ATCC 43890 strain culture in LB broth the day before analysis. Thus, the concentration of paramagnetic gadobutrol was 0.2M in nutrient broth. The syringe with E. coli ATCC 43890 strain culture and paramagnetic nutrient medium was inserted in magnetic bioprinter (FIG. 1) on the next day. There were no macroscopic changes in bacterial distribution by volume during the first hour of observation (FIG. 2A). Peripheral regions clearance was observed two hours later. Further observations have shown that clearance is conceivably associated with the slow movement of bacteria from the periphery to the centre. The peripheral regions were clean 9 hours after the start of the experiment, while the cuvette centre was filled with rather large sphere of bacteria (FIG. 2A). This sphere has decreased in volume 24 hours after the start of the experiment (compared to its size on 9 hours time mark) but it was still rising in the syringe, while other regions were 100% clear and seemed to be bacteria free. These observations have shown that bacteria can not leave the magnetic trap. Prolonged incubation did not cause any further changes, it shows that all the forces acting on the bacteria were balanced. The same experiment with 0.1M gadobutrol was carried out. Using lower gadobutrol concentrations has shown that the process was identical, although the diameter of the sphere 24 hours after was larger and its position was closer to the bottom of the syringe (FIG. 2B).

Then the experimental conditions were changed to ensure bacterial growth. E. coli ATCC 43890 culture was diluted in a ratio of 1:100 by the paramagnetic medium containing 0.1M gadobutrol the day before. The next day the culture was placed in the bioprinter and incubated at 37° C. for 24 hours (FIG. 2C). Then concentration of gadobutrol was increased to 0.2M. There was the small sphere in the centre of the syringe filled with paramagnetic medium (the medium with 0.2M gadobutrol) in 24 hours after the start of the experiment, whereas, peripheral areas were free from bacteria (FIG. 2D).

The behavior of Gram-negative and Gram-positive bacteria grown in the magnetic bioprinter was compared. Three E. coli strains including laboratory strain JM109, probiotic strain M-17 and Shiga-toxin producer strain ATCC 43890 were used as models of Gram-negative bacteria. Staphylococcus aureus and Listeria monocytogenes have been used as models of Gram-positive bacteria. Bacteria were cultivated in paramagnetic nutrient medium containing 0.2M gadobutrol placed in the magnetic bioprinter for 24 hours. All the tested bacteria formed spheres levitating in the column of fluid (FIG. 3). The geometric parameters of these spheres were different depending on the strain (FIG. 3B). The sphere formed by the laboratory strain JM109 was significantly larger than spheres formed by the pathogenic strain ATCC 43890 and the probiotic strain M17 (p<0.05). E. coli ATCC 43890 and S. aureus formed oblong spheres, while E. coli M-17 and especially L. monocytogenes formed more circular spheres.

### Example 2. Estimation of Bacteria Survivability in Magnetic Trap

Bacterial Survivability Test

Bacterial survivability in aggregates was determined by staining cells with a set of molecular probes LIVE/DEAD BacLight Bacterial Viability Kit (Molecular Probes). Bacteria were cultivated for three days in proper paramagnetic nutrient medium in magnetic field. The aggregates were washed three times with PBS and were destroyed by shaking for 30 seconds to form the cell suspension three days after in vitro cultivation. The cell suspensions were incubated for 15 minutes in PBS containing fluorescent dye SYTO™ 9 (2.5 μM) and propidium iodide (4 μM). Stained cells were observed using the Eclipse Ti-E microscope with confocal A1 module (Nikon Corporation, Japan) and CFI Plan Apo VC 20×/0.75 objective lens. The neural network U-Net with convolutional architecture was used for quantitative estimation of vital and dead bacteria (considering loss function with Dice measure) [Ronneberger, O. et al. U-net: Convolutional networks for biomedical image segmentation. in Lecture Notes in Computer Science (including subseries Lecture Notes in Artificial Intelligence and Lecture Notes in Bioinformatics) (2015)]. The network was educated on 100 microscopic images of microorganisms that were processed by human. The network prediction represented the matrix with probability of microorganism location for each pixel of the original image. Areas with the probability higher than 0.98 were later counted by the Suzuki-Abe algorithm [Suzuki, S. & be, K. A. Topological structural analysis of digitized binary images by border following. Comput. Vision, Graph. Image Process (1985)].

Cell Mobility Analysis

The mobility analysis was performed in the following manner. E. coli cultures were cultivated in standard Petri dishes in LB during the night, then they were used for culturing in 50 ml tubes (syringes) filled with LB broth. Culturing was performed using inoculating needles. Mobility was estimated as the average spot diameter measured at different depths 16 hours after culturing. The data was taken from each of the three experiments and was used to calculate mean values and standard deviation.

The E. coli ATCC 43890 (cultured in different conditions) concentrations were estimated for analysis of survivability of bacteria in presence of gadobutrol. Primary culture has shown concentrations of 2.3×10⁹, 2.1×10⁹ and 1.9×10⁸ cfu/ml for fresh night culture grown in LB broth during the night (1), for culture grown in LB broth during the night and then incubated with 0.2M gadobutrol in the bioprinter for next 24 hours (2), and culture grown under magnetic levitation in the paramagnetic nutrient medium (3) respectively (FIG. 4A). The obtained results have shown that bacteria saved their survivability after incubation with 0.2M gadobutrol. Moreover, the results have shown that the bacteria proliferated in the paramagnetic culture medium, that was observed due to the increase in concentration during cultivation: the initial bacteria (diluted in fresh paramagnetic medium) concentration was 2.3×10⁷ cfu/ml ( 1/100 from the fresh night culture) and final concentration was 1.9×10⁸ cfu/ml.

The analysis of bacterial cultures has shown that small spheres formed by E. coli ATCC 43890 and grown in the magnetic bioprinter had at least 10 times less colony forming units in comparison with night culture grown under standard conditions on mixer (FIG. 4A). Such difference can be caused by limited growth in the magnetic trap or death of the part of bacterial population due to magnetic levitation conditions. Differential staining (Live/Dead Assay technique) was applied to bacterial spheres formed by E. coli ATC43890 and other strains under magnetic levitation conditions to detect dead cells (FIG. 4B). Approximately 16% of ATCC 43890 bacteria were dead. Dead cells were revealed in all bacterial spheres, but their percentage varied from strain to strain (FIG. 4C). L. monocytogenes has shown the best results: just 0.77% of dead cells. The results for ATCC 43890 and JM109 were the worst: 16.58% and 14.3% of dead cells respectively. However, this was just a small percentage of the total population. It supports the idea of bacterial growth restrictions in magnetic trap besides direct mortality.

### Example 3. Electron Microscopy Visualization of Structures Grown in the Magnetic Trap has Revealed Aggregates with Characteristics Typical for Biofilms

Biofilm-Like Aggregates Visualization

Bacteria grown in the magnetic trap were fixed with 2.5% glutaraldehyde and were washed with PBS 3 times for 10 minutes. Aggregates structure was analyzed via confocal laser scanning microscopy and scanning electron microscopy (CLSM and SEM). Cell nucleoids were visualized with SYBR Green I stain. Biofilm matrix elements were visualized with Film Tracer™ SYPRO® Ruby Biofilm Matrix Stain. The confocal microscope Zeiss LSM 510Meta (Carl Zeiss, Germany) and Plan-Apochromat 63/1.4 Oil DIC objective lens were used for imaging. Images were processed using Zeiss LSM software 510Meta version 3.2. Aggregates samples for SEM were prepared according to the standard procedure used for biofilms [Maricarmen Iñiguez-Morenoa et al. Int J Food Microbiol, 303, 32-41 (2019)], they were spray-coated with 20 nm thick layer of platinum. The Camscan S2 scanning electron microscope (Cambridge Instruments, Great Britain) in SEI mode with optical resolution of 10 nm and operating voltage of 20 kV was used for imaging. Images were obtained using MicroCapture software (SMA, Russian Federation).

Initial attempts to produce spheres of aggregated bacteria in the magnetic bioprinter have shown that the bacterial spheres incubated for 24 hours were fragile when removed from the syringe. Incubation was extended up to 5 and 7 days. The 5- and 7-days spheres were not as fragile and supported macroscopic aggregates which maintained their structure. This suggests that the bacteria were somehow linked. The most fragile sphere that broke in pieces was formed by the laboratory strain E. coli JM109. The most stable spheres which broke in larger fragments or did not break and maintained its stable macroscopic three-dimensional structure for a long time were formed by E. coli ATCC 43890 and S. aureus.

The 7-days sphere formed by E. coli ATCC 43890 and grown under magnetic levitation conditions was fixed with glutaraldehyde and removed from the syringe. Then all the remaining aggregates were undergone microscopic studies. The fixed sample was marked with fluorescent dye SYBR Green (binding nucleic acid) and Film Tracer™ SYPRO® Ruby Biofilm Matrix Stain and examined with confocal laser scanning microscopy (CLSM). SYBR Green that mainly stains nuclear DNA has demonstrated the presence of multiple single bacterial cells (FIG. 5A). Some cells were elongated and reached 4- or 5-fold length of a normal cell. Bacterial cells were fixed in matrix stained with Ruby Biofilm Matrix Stain to form three-dimensional structure (FIG. 5B).

E. coli ATCC 43890 strain was studied via scanning electron microscopy for better understanding of the morphology of bacterial aggregates formed under magnetic levitation conditions. The observed three-dimensional structure has confirmed that the aggregates were formed by bacterial cells and the extracellular matrix produced by themselves. CLSM has helped us to reveal long bacterial cells surrounded by the extracellular matrix inside the aggregates, and SEM has shown short and nearly oval cells surrounded by the extracellular matrix.

### Example 4. Curli Protein which is Strictly Necessary for Fabrication of Two-Dimensional Attached E. coli Biofilms was of No Importance for Fabrication of Biofilm-Like Aggregates Under Magnetic Levitation Conditions

Similarities between levitating aggregates produced in the magnetic trap and two-dimensional attached biofilms suggest that aggregates fabrication may be caused by the similar mechanisms as biofilms fabrication. E. coli is one of the most well studied models in terms of biofilm fabrication. Size and stability of the aggregates formed in the magnetic trap were different for different E. coli strains used in this study as illustrated above. The fabrication of two-dimensional attached biofilms on abiotic surfaces with three E. coli strains was studied for further comparison of levitating aggregates with two-dimensional attached biofilms.

Microplate Assay of Biofilms Fabrication Dynamics

The standard microtiter plate assay was used for attached biofilm fabrication analysis [Merritt, J. H., Kadouri, D. E. & O'Toole, G. A. Curr. Protoc. Microbiol. (2011)]. The E. coli culture incubated over night was diluted 1:100 in fresh LB broth and incubated in a 96 well microtiter plate for 48 hours. Then the wells were washed with PBS and stained with 0.1% solution of crystal violet for 10 minutes. The dye was solubilized by adding 95% ethanol to each stained well to evaluate the biofilm biomass. Then the solution was transferred to 96-well flat-bottomed plate for further optical density measurement on 500 nm wavelength via iMark spectrophotometer (BioRad).

Analysis of Curli Protein Production

The binding assay of Congo red (CR) was performed for revealing of curli protein production [Reichhardt, C. et al. PLoS One (2015)]. Congo red was added to LB agar up to final concentration of 25 μg/ml. The bacteria were cultured and incubated for 24 hours at 37° C. Binding was detected as a red staining of the bacterial culture due to the binding of Congo red with the curli amyloid structures.

First, we compared the fabrication of two-dimensional attached biofilms by various E. coli strains. The standard microtiter plate assay was used to evaluate the efficiency of biofilm fabrication. The pathogenic ATCC 43890 strain was the worst in terms of biofilm fabrication in this test (FIG. 6A). This strain did not form representative biofilms even after 48 hours and under standard conditions. Two other E. coli strains JM109 like M-17 have formed biofilms in a more efficient way. Then we compared bacteria movements and production of curli protein. It was shown in earlier studies that the mobility itself, flagellum and curli protein are essential for E. coli biofilm fabrication (Pratt [symbol] Kolter, 1998; Prigent-Combaret et al., 2000; Wood et al., 2006). Studies of bacterial growth in semisolid agar showed that the JM109 strain was least mobile among the other three strains while the ATCC 43890 strain was most mobile (FIG. 6B). The binding assay of Congo red was performed to analyze the curli protein production. JM109 and M-17 strains have bound Congo red effectively, that allows us to confirm their production of curli protein (FIG. 6C). The pathogenic ATCC 43890 strain did not bind the Congo red, so it does not have curli protein on its surface. The latest result corresponded with low biofilm production by this strain in the microtiter model. However, the ATCC 43890 strain effectively formed aggregates in the magnetic trap.

The obtained data points to the fact that the ability to form three-dimensional non-attached biofilm-like aggregates and the ability to form biofilms on the abiotic surfaces are not always fully correlated. It is rather due to coincidence than due to identical fabrication mechanisms.

Therefore, the method of non-attached microorganism biofilms (biofilm-like aggregates) fabrication under in vitro conditions has been developed. It has been illustrated that various microorganisms (including but not limited to Gram-negative and Gram-positive bacteria) form three-dimensional non-attached biofilm-like aggregates. These biofilm-like aggregates are not attached to any surfaces or other substrates, have a three-dimensional structure formed by microorganisms (such as bacteria) and extracellular matrix. Microorganisms growth is the essential aspect for aggregates fabrication. The produced biofilm-like aggregates have high stability and survivability. Experiments have shown that bacteria survivability varies from 83.4% to 99.7% depending on the strain after 3-day growth. The produced biofilm-like aggregates are similar in their properties to biofilms formed by microorganisms in natural conditions. It has been shown that the biofilm-like aggregates fabrication by this method is happening due to the microorganisms growth and reproduction. This aspect is similar to microcolonies formation as the key stage of biofilm fabrication.

Biofilm-like aggregates formed by E. coli ATCC 43890 strain were examined via CLSM and SEM. We have revealed that these aggregates were three-dimensional structures formed by microorganisms and extracellular matrix. The extracellular matrix was the product of microorganisms (along with they growth and development) rather than being formed by medium proteins. Some E. coli cells in biofilm-like aggregates were elongated. SEM analysis has confirmed the CLSM analysis results and has demonstrated the vesicular morphology of the matrix. Comparison of two-dimensional attached biofilms and non-attached biofilm-like aggregates of this invention revealed the difference in the mechanisms underlying their fabrication. In particular, curli protein which is required to form two-dimensional attached E. coli biofilms was not required to form non-attached aggregates.

Further advantages of the method from this invention (over current ones) are the absence of any limitations to the equipment size and composition of the medium. Moreover, the macroscopic size of produced biofilm-like aggregates allows to perform monitoring of aggregate growth processes in real time. Scaling the biofilm production process is possible with the use of magnetic system providing continuous supplement/inflow of paramagnetic medium which can additionally include antibiotics, special markers or other components depending on the current objectives. The suggested simple scheme can be useful for simulating of different microorganisms behavior, the influence of various conditions and injection of tested medications on microorganisms development. For example, it is possible to simulate the bacteria behavior at chronic infections such as chronic bronchitis, otitis or rhinosinusitis, in patients without CF and corresponding mucus accumulation; as well as to simulate the microorganisms growth under microgravity conditions.

The invention has been described with the references to the disclosed embodiments, thus it should be clear for those skilled in the art that such detailed experiments are presented only for illustration of this invention, they should not be considered as confining the scope of the invention in any way. It is clear that implementation of various modifications is possible without departing from the scope of the present invention. Thus, the suggested method can be successfully used to fabricate biofilm-like aggregates of such microorganisms as protozoa, fungi, microalgae, Gram-positive or Gram-negative bacteria or its consortiums without any significant modifications.

1. Method for producing of non-attached biofilm-like microorganisms aggregates including cultivation of stated microorganisms under magnetic levitation conditions in inhomogeneous magnetic field.

2. The method of claim 1 wherein the microorganisms are cultivated in central part of inhomogeneous magnetic field with the lowest field intensity.

3. The method of claim 1 wherein the inhomogeneous magnetic field is created using a magnetic system consisting of at least two annular permanent magnets oriented towards each other by the same poles.

4. The method of claim 1 wherein the inhomogeneous magnetic field is created using Bitter magnets.

5. The method of claim 1 wherein the inhomogeneous magnetic field is created using superconducting magnets.

6. The method of claim 1 wherein the microorganisms can be protozoa, fungi, microalgae, Gram-positive or Gram-negative bacteria and/or its consortiums.

7. The method of claim 1 wherein the microorganisms are placed in the inhomogeneous magnetic field in the cultivation vessel in suspension on paramagnetic substrate (which is the substrate with paramagnetic for cultivation of microorganisms).

8. The method of claim 7 wherein the paramagnetic properties of the substrate are provided by the presence of gadolinium.

9. The method of claim 8 wherein the gadolinium is added to the medium as gadobutrol.

10. The method of claim 7 wherein there is inflow of paramagnetic medium to the cultivation vessel as microorganisms growing.

11. The method of claim 1 wherein the cultivating environment is adjusted according to the chosen microorganisms to create the optimal conditions and/or study the influence of the cultivating environment on biofilm-like microorganisms aggregates fabrication.

12. The method of claim 7 wherein the test compounds, biologically active substances, medications and/or mixtures.

13. Biofilm-like microorganisms aggregate produced by any of the methods of claim 1-12.

14. Biofilm-like microorganisms aggregate on 13 where microorganisms may present as protozoa, fungi, microalgae, Gram-positive or Gram-negative bacteria and/or its consortiums.
